(19) United States
(12) Patent Application Publication
(10) Pub. No.: US 2026/0224510 A1
(43) Pub. Date: Aug. 6, 2026

(54) COMPOSITIONS AND METHODS FOR THE PREVENTION AND MANAGEMENT OF RADIATION DERMATITIS AND HAND-FOOT SYNDROME

(71) Applicant: RIDALES AB, Johanneshov (SE)

(72) Inventor: Rahma WEHELIE, Johanneshov (SE)

(21) Appl. No.: 19/149,962

(22) PCT Filed: Jul. 22, 2024
(86) PCT No.: PCT/SE2024/050053

§ 371 Date: Jul. 22, 2025

(30) Foreign Application Priority Data
Jan. 23, 2023 (SE) 2350057-2

(51) Int. Cl. A61K 31/045; A61K 9/00; A61K 31/716; A61K 36/185; A61K 36/886; A61P 17/02; A61P 17/16

(52) U.S. Cl. CPC A61K 31/045; A61K 9/0014; A61K 31/716; A61K 36/185; A61K 36/886; A61P 17/02; A61P 17/16

(57) ABSTRACT

The present invention relates to a composition for topical administration comprising a colloidal oatmeal and a 4-t-butylcyclohexanol, wherein the colloidal oatmeal comprises β-glucan and optionally avenanthramide. The 4-t-butylcyclohexanol is in an amount in the range of 0.1-5 wt % of the total amount of the composition, and the β-glucan is in an amount in the range of 1-10 wt % of the total amount of the composition. The present invention further relates to said composition for topical administration for use in therapy and said composition for topical administration for use in treating or preventing a skin condition associated with cancer radiation therapy or chemotherapy, and also the use of said composition in treating or preventing a skin condition associated with cancer radiation therapy or chemotherapy.

### FIELD OF THE INVENTION

The field of the invention is compositions and methods for preventing and managing radiation dermatitis and hand-foot syndrome in cancer patients, who receive treatment in the form of radiation therapy and/or chemotherapy. The compositions and methods may also be beneficial in the treatment of wound healing caused by chemotherapy and radiation treatments in general.

### TECHNICAL BACKGROUND

Cancer diagnosis is on the rise worldwide, from approximately 18.1 million new cases worldwide in 2020 to an estimated 27.5 million new cancer cases by 2040 will be diagnosed (see Worldwide cancer statistics | Cancer Research UK, accessed Dec. 21, 2021). Approximately 50% of these patients will be prescribed radiation therapy. Radiation therapy is effective in increasing survival rates and providing palliative care, (see e.g. Baskar R, et al. Biological response of cancer cells to radiation treatment, Front Mol Biosci. 2014 Nov. 17; 1:24. doi: 10.3389/fmolb.2014.00024. PMID: 25988165; PMCID: PMC4429645). Radiation therapy is meant to destroy cancer cells, while minimizing the effect on normal cells. Radiation therapy also has the added advantage that it is cost effective, representing only approximately 5% of the cost of treating cancer, (see e.g. Ringborg U., et al. 2003). The Swedish Council on Technology Assessment in Health Care (SBU) systematic overview of radiotherapy for cancer including a prospective survey of radiotherapy practice in Sweden, for the 2001-summary and conclusions, see Acto. Oncol. 42, 357-365. 10.1080/02841860310010826 [PubMed]. However, the beneficial effects of radiation therapy are tainted by some of its side effects, the most common one being radiation dermatitis.

Radiation dermatitis occurs most frequently in patients who receive radiation therapy for breast, cervix, prostate, eye, head and neck cancers or cancers that develop on or near skin, such as skin or anal cancer.

Radiation dermatitis or radiations burn involves reddening, itchy skin, dry desquamation and peeling skin, swelling, blistering, and/or open sores that may appear where the skin is sweaty or damp, such as the armpits or under the breasts, (see e.g. Radiation Burn (Dermatitis): What it is, Treatment, Management, clevelandclinic.org). In severe cases, skin breakage can occur, resulting in moist desquamation or pain to open bleeding ulcers, which can greatly affect the quality of life of patients. Severe acute radiation dermatitis can lead to interruption or delay of treatment specially radiation treatments with dose threshold from Gy-50 Gy or higher doses.

Although the intensity and severity of the condition can vary greatly, depending on the target of Radiation Therapy (RT) and the dose delivered, radiation dermatitis (RD) can significantly influence patients' quality of life during and after completion of RT. Strategies to prevent or mitigate RD are aimed at improving comfort during RT, reducing the degree of inflammation, and promoting healing of skin areas with more severe dermatitis. Corticosteroids, such as cortisone, especially in the form of topical creams, have been used in a number of cases to relieve itching. However, long-term use of corticosteroids has led to local and systemic adverse events, and thus, their use should be avoided, if at all possible, (see e.g. Sobhan, Mohammadreza, et al. “The Efficacy of Colloidal Oatmeal Cream 1% as Add-on Therapy in the Management of Chronic Irritant Hand Eczema: A Double-Blind Study.” Clinical, Cosmetic and Investigational Dermatology, vol. 13, 2020, pp. 241-251. p. 247, https://doi.org/http://doi.org/10.2147/CCID.S246021). While a wide variety of topical, oral, and intravenous agents are used to prevent/treat RD, no treatment can be explicitly recommended because currently there is no gold standard in the prevention and management of this condition.

Palmar-plantar erythrodysesthesia, also known as hand-foot syndrome (HFS) is a distinctive and relatively frequent dermatological toxic reaction associated with certain chemotherapeutic agents and is widely recognized in determining dose-limiting toxicity of certain chemotherapy agents, specifically pegylated liposomal doxorubicin, doxorubicin, capecitabine, mitotane, cyclophosphamide, 5-fluorouracil, cytarabine, docetaxel, paclitaxel, sorafenib, sunitinib and gefitinib, (see e.g. Pereira, Paulina Patente, et al. “Identification, Prevention and Treatment of Hand-Foot Syndrome Induced by Chemotherapy: Systematic Review” Revista Brasileira De Cancerologia, vol. 65, no. 4, 2019, pp. 35-46; Inokuchi, Masafumi, et al. “Treatment of Capecitabine-Induced Hand-Foot Syndrome Using a Topical Retinoid: A Case Report.” Oncology Letters, vol. 7, 2014, pp. 444-448, https://doi.org/10.3892/ol.2013.1706). The manifestations of HFS have been classified into 3 grades according to their severity. HFS (of all grades) occurred in ~50% of patients in receiving chemotherapy treatment for metastatic breast or colorectal cancers or other cancer forms, with 10% of patients experiencing severe HFS (grade 3). The side effects on hands and feet include redness, dry flaky skin and swelling on the hands and soles of the feet, which can develop into blisters, cracks and sores. Patients may also feel sore and have tingling and numbness in their hands and feet. Although this cutaneous adverse effect of chemotherapy was first described almost 30 years ago, it has become an increasingly important adverse effect, due to the chemotherapy drugs that are now commonly used. Despite this, the pathogenesis of this disorder remains unknown. No effective preventative treatment has been definitively established, thus necessitating chemotherapy dose reduction in severe cases.

The need remains for an effective prevention and management of these frequent conditions associated with cancer treatments in the form of radiation therapy and chemotherapy.

### SUMMARY OF THE INVENTION

In view of the above, an object of the present invention is to provide a composition for topical administration, or a dermatological composition, that is designed to alleviate and help to heal wounds of the skin or other skin conditions associated with cancer radiation therapy, chemotherapy or sunburn. Preferably, the topical or dermatological composition is designed to alleviate and help to heal wounds of the skin or other skin conditions associated with cancer radiation therapy or chemotherapy.

Another object is to provide a composition for topical administration, or a dermatological composition, that is designed to be used as a pre-treatment and post-treatment, before and after radiation therapy, such as treatment of breast cancer by radiation therapy.

Another object is to provide a composition for topical administration, or a dermatological composition, that is designed to be used to alleviate and heal skin conditions which may be caused by chemotherapy, such as hand-foot syndrome.

To achieve at least one of the above objects and also other objects that will be evident from the following description, a composition for topical administration having the features defined in claim 1 is provided according to the present inventive concept. A composition for use in therapy, as well as a composition for use in treating or preventing a skin condition associated with cancer radiation therapy or chemotherapy, are provided in the independent claims. Preferred variations to the inventive concept will be evident from the dependent claims.

According to a first aspect of the invention, there is provided a composition for topical administration comprising a colloidal oatmeal and a 4-t-butylcyclohexanol, wherein the colloidal oatmeal comprises β-glucan and optionally avenanthramide, wherein the 4-t-butylcyclohexanol is in an amount in the range of 0.1-5 wt % of the total amount of the composition, and wherein the β-glucan is in an amount in the range of 1-10 wt % of the total amount of the composition.

In an alternative embodiment, there is provided a composition for topical administration comprising a colloidal oatmeal and a 4-t-butylcyclohexanol, wherein the colloidal oatmeal comprises at least one of β-glucan and avenanthramide. The colloidal oatmeal may comprise β-glucan in the range of 1-10 wt % preferably in the range of 1-5 wt %, or avenanthramide in an amount in the range of 0.5-5 wt %, respectively. The colloidal oatmeal may comprise β-glucan in an amount in the range of 1-10 wt %, preferably in the range of 1-5 wt %. The colloidal oatmeal may comprise avenanthramide in an amount in the range of 0.5-5 wt %. Advantageously, the colloidal oatmeal and/or the avenanthramide have anti-inflammatory and antioxidant properties, while the 4-t-butylcyclohexanol relieves itching and burning sensations and is a non-steroidal alternative to cortisone cream.

The colloidal oatmeal may be in an amount in the range of 1-10 wt % of the total amount of the composition, preferably in the range of 1-5 wt %.

Additionally, or alternatively, the composition may comprise the 4-t-butylcyclohexanol is in an amount in the range of 0.1-5 wt % of the total amount of the composition, preferably in the range of 0.2-4.0 wt %, more preferably in the range of 0.3-3.0 wt %, even more preferably in the range of 0.5-2.5 wt %, even more preferably in the range of 0.6-2.0 wt %, or most preferably in the range of 0.5-2.0 wt %. The addition of the 4-t-butylcyclohexanol is specifically to reduce stinging associated radiation treatments with dose threshold from 10 Gy-50 Gy or higher doses and chemotherapy treatments.

In yet another embodiment, the composition according to any of the previous embodiments may further comprise additional β-glucan, which may be in the form of a liquid or powder. The additional β-glucan may be in an amount in the range of 1-10 wt % of the total amount of the composition, preferably in the range of 1-5 wt %. The additional β-glucan may increase the anti-inflammatory and antioxidant properties of the composition, at times where such an increase is warranted, e.g. during radiation therapy. It has also surprisingly been found that the specific combination of the components of the composition of the present invention, e.g. 4-t-butylcyclohexanol and β-glucan may prevent and/or assist in the healing procedure of the skin which have subjected to radiation therapy or chemotherapy.

In yet another embodiment, the composition according to any of the previous embodiments may further comprise at least one of Aloe vera whole leaf extract, Butyrospermum Parkii Shea oil, avenanthramides, white petrolatum, vitamin E, Jojoba oil, and piroctone olamine.

In yet another embodiment, the composition according to any of the previous embodiments may be in the form of a lotion, gel, cream, ointment, or spray. One of the advantages of using these alternatives is future production of the product.

In yet another embodiment, the composition according to any of the previous embodiments may further comprise additional avenanthramide. The additional avenanthramide may be in an amount in the range of 0.5-5 wt % of the total amount of the composition. The additional avenanthramide may increase the anti-inflammatory and antioxidant properties of the composition, at times where such an increase is warranted, e.g. during radiation therapy.

In yet another embodiment, the composition according to any of the previous embodiments may further comprise one or more additional components selected from dermatologically acceptable carriers, preservatives, surfactants, humectants, emulsifiers, thickening agents, perfumes, preservatives, vegetable or mineral oils, antiseptic agents, acidifying or alkalinizing agents, vitamins, antioxidants, anti-UV agents, solvents, pH-stabilizing agents, silicones and combinations thereof.

In yet another embodiment, the composition according to any of the previous embodiments may comprise 1-10 wt % colloidal oatmeal; 0.1-5 wt % 4-t-butylcyclohexanol; optionally 1-10 wt % β-glucan; 0.1-5 wt % piroctone olamine; 0.1-5 wt % Aloe vera whole leaf extract; 0.1-2 wt % Butyrospermum Parkii Shea oil; optionally 0.5-5 wt % avenanthramides; 0.1-20 wt % white petrolatum; and 20-60 wt % water.

In yet another embodiment, the composition according to any of the previous embodiments may comprise 1-10 wt % colloidal oatmeal; 0.1-5 wt % 4-t-butylcyclohexanol, preferably 0.5-2.0 wt %; 4-t-butylcyclohexanol; 1-10 wt % β-glucan, preferably 1-5 wt % β-glucan; 0.1-5 wt % piroctone olamine; 0.1-5 wt % Aloe vera whole leaf extract; 0.1-2 wt % Butyrospermum Parkii Shea oil; optionally 0.5-5 wt % avenanthramides; 0.1-20 wt % white petrolatum; and 20-60 wt % water.

In a second aspect of the present invention relates to the composition according to the first aspect for use in therapy.

In a third aspect the composition according to the first aspect may be for use in treating or preventing a skin condition associated with cancer radiation therapy, chemotherapy or sunburn. Preferably the composition according to the first aspect may be for use in treating or preventing a skin condition associated with cancer radiation therapy or chemotherapy.

The composition according to the first aspect may be for use in treating or preventing a skin condition associated with cancer radiation therapy. Preferably the skin condition may be radiation dermatitis.

In another embodiment the composition according to the first aspect may be for use in treating or preventing a skin condition associated with chemotherapy. Preferably the skin condition may be hand-foot syndrome.

In a fourth aspect there is provided a use of the composition according to the first aspect in treating or preventing a skin condition associated with cancer radiation therapy or chemotherapy.

The use of a composition for topical administration according the fourth aspect, wherein the skin condition is selected from the hand-foot syndrome, blisters, dry or moist desquamation, redness, dryness, peeling of the skin, itching, stinging, burning, erythema, atopic dermatitis, psoriasis, inflammation, irritation and radiation dermatitis. The cancer may be breast cancer, brain cancer, neck cancer, skin cancer, lymphoma, prostate cancer or colorectal cancer.

In an embodiment of the third aspect the skin conditions may be selected from hand-foot syndrome, blisters, dry or moist desquamation, redness, dryness, peeling of the skin, itching, stinging, burning, erythema, atopic dermatitis, psoriasis, inflammation, irritation, radiation dermatitis and allergy, preferably the skin conditions may be selected from hand-foot syndrome, blisters, dry or moist desquamation, redness, dryness, peeling of the skin, itching, stinging, burning, erythema, atopic dermatitis, psoriasis, inflammation, irritation and radiation dermatitis, even more preferably the skin conditions may be selected from hand-foot syndrome and radiation dermatitis. In one specific embodiment the skin condition may be hand-foot syndrome. In another specific embodiment the skin condition may be radiation dermatitis.

The composition for use in treating or preventing a skin condition associated with cancer radiation therapy according to the third aspect, wherein the radiation treatment dose may be at least 10 Gy, at least 20 Gy, at least 30 Gy, at least 40 Gy or at least 50 Gy or a higher dose, preferably the radiation treatment dose may be in the range of 10 Gy-50 Gy. Another preferred radiation treatment dose may be in the range of 10 Gy-16 Gy.

In a fourth aspect there is provided a use of the composition according to the first aspect in treating or preventing a skin condition associated with cancer radiation therapy or chemotherapy.

In another aspect of the present invention there is provided a method of producing a composition for topical administration according to the first aspect comprising the steps of: providing colloidal oatmeal and a 4-t-butylcyclohexanol, wherein the colloidal oatmeal comprises at least one of β-glucan and avenanthramide; and optionally adding additional components selected from dermatologically acceptable carriers, emollients, fragrance, thus providing the composition, preferably in the form of a lotion, cream, gel or ointment. In another aspect of the present invention there is provided a method of producing a composition for topical administration according to the first aspect comprising the steps of: providing a colloidal oat, β-glucan and a piroctone olamine; mixing the colloidal oat, β-glucan and the piroctone olamine; and optionally adding additional components selected from dermatologically acceptable carriers, emollients, fragrance, thus providing the composition, preferably in the form of a lotion, cream, gel or ointment.

In one embodiment there is provided there is provided a method of producing a composition for topical administration according to the first aspect comprising the steps of: providing β-glucan, avenanthramide and 4-t-butylcyclohexanol; and optionally adding additional components selected from dermatologically acceptable carriers, emollients, fragrance, thus providing the composition, preferably in the form of a lotion, cream, gel or ointment.

### BRIEF DESCRIPTION OF DRAWINGS

FIG. 1 shows a typical hand-foot-syndrome patient's hands following a second cycle of chemotherapy.

FIG. 2 shows inventor's hands (left and right) following a first cycle with the same type of chemotherapy agents as used for the patient in FIG. 1.

FIG. 3 shows common skin reactions in patients receiving breast radiation therapy with dose threshold from 10 Gy-50 Gy or higher doses, (A) Follicular reaction with pruritus, (B) Skin erythema and edema, (C) Dry desquamation in axillary fold, and (D) Moist desquamation in inflammatory fold.

FIG. 4 shows the inventor's skin (A) that was subject to 50 Gray (Gy) radiation therapy (RT) at 4 weeks following the end of the radiation and, (B) after using the composition according to the invention for 5 days after RT, and (C) continued using the composition according to the invention for 3 months after RT.

### DETAILED DESCRIPTION OF THE INVENTION

The inventor (Rahma Wehelie, PhD) was diagnosed triple negative breast cancer that had spread to lymph nodes. As treatment for the breast cancer, the inventor was prescribed chemotherapy to shrink the size of the cancer tumor, to be followed by mastectomy and radiotherapy.

### Chemotherapy and Hand-Foot Syndrome

For the chemotherapy part of her cancer treatment, the inventor received chemotherapy. Many chemotherapy agents are known to cause HFS which are redness, dry flaky skin and swelling on the hands and soles of the feet. FIG. 1 shows a typical HFS patient's hands following a second cycle of paclitaxel and capecitabine; the patient shows extensive hyperpigmentation and desquamation of hands, see e.g. https://ijdvl.com/hand-and-foot-syndrome-secondary-to-capecitabine/. The inventor developed mild blisters on her hands and feet. Since no effective preventative or treatment has yet been established for HFS, the inventor experimented and invented a cream, the topical composition according to the present inventive concept, for the treatment of Hand Foot Syndrome.

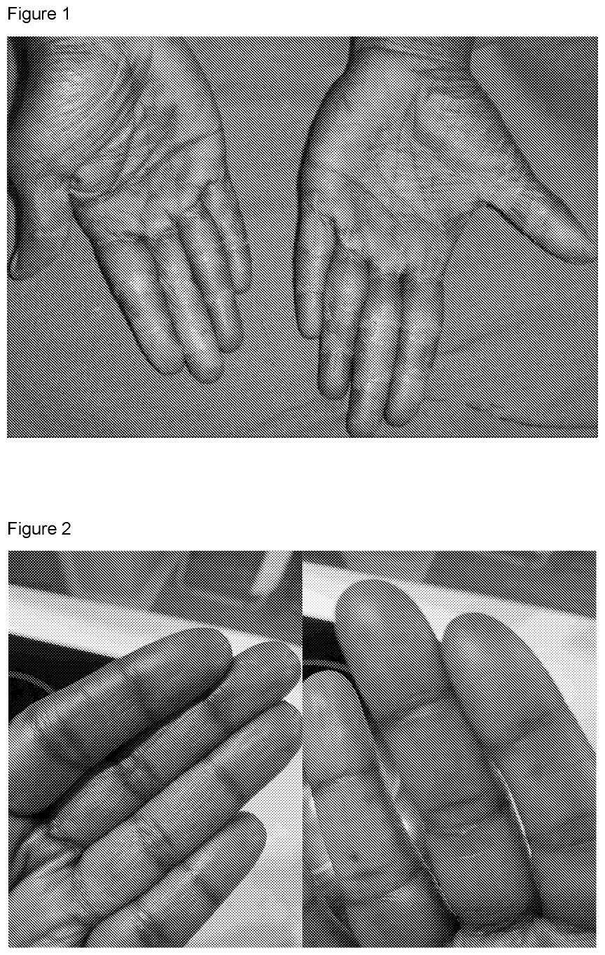

The inventor treated herself daily until blisters on her hands and feet were back to normal with colloidal oatmeal or Avenacare Eco Oat Beta Glucan (Lantmännen). The β-glucan that was used in the composition was in the range of 1-5 wt % of the total amount of the composition. In some examples the β-glucan was 1 wt %, 2 wt %, 3 wt %, 4 wt % or 5 wt %. The optimum amount was found at 3 wt % of the total amount of the composition. The colloidal oatmeal was mixed with 4-t-butylcyclohexanol, which was in the range of 0.5-2.0 wt % of the total amount of the composition. In some examples the 4-t-butylcyclohexanol was 0.5 wt %, 1 wt %, 1.5 wt % or 2 wt %. Aloe vera whole leaf extract gel, Butyrospermum Parkii (Shea) Oil in a form liquid, Jojoba oil, Vitamin E and white petrolatum gel were also mixed into the composition. In a preferred embodiment the amount of β-glucan was 3 wt % and the amount of 4-t-butylcyclohexanol was 2 wt %. FIG. 2 shows inventor's hands following a second cycle with the same type of chemotherapy agents.

### Radiotherapy and Radiation Dermatitis

Radiotheraphy or radiation therapy is a treatment using ionizing radiation and it is generally provided as part of cancer therapy to either kill or control the growth of malignant cells. It is normally delivered by a linear particle accelerator. Radiation therapy may be curative in a number of types of cancer if they are localized to one area of the body, and have not spread to other parts. It may also be used as part of adjuvant therapy, to prevent tumor recurrence after surgery to remove a primary malignant tumor, e.g. in early stages of breast cancer. Radiation therapy is synergistic with chemotherapy, and has been used before, during, and after chemotherapy in susceptible cancers. One of the major problems with radiation therapy is that the ionizing radiation consisting of subatomic particles or electromagnetic waves that have sufficient energy to ionize atoms or molecules by detaching electrons from them. The subatomic particles or electromagnetic waves are on the high-energy portion of the electromagnetic spectrum, which means the higher energy ultraviolet part of the electromagnetic spectrum is ionizing radiation, preferably electric magnetic radiation in the >10 MeV region. This is of course beneficial in the aspect of killing or controlling the growth of malignant cells. However, this also leads to harmful reactions of healthy tissue and thus high exposure, or doses of ionizing energy leads to radiation burns, or development of radiation dermatitis. Simultaneously, chemotherapy therapy often leads to the development of hand-foot syndrome. For example, for an exposure with an energy of 1 MeV, the dose in averaged human tissue will be 1 radiation absorbed dose (rad). The absorbed dose is a term used to describe how much energy of the radiation that deposits in a material, the absorbed dose may also be measured in Gray or Gy, which is defined as the absorption of one joule of radiation energy per kilogram of matter. The equivalent dose calculates the effect of radiation on human tissue. For curative purposes, the typical dose for a solid epithelial tumor may be in the range of 60-80 Gy, while lymphomas may be treated with 20-40 Gy. Preventive or adjuvant doses are typically around 45-60 Gy in 1.8-2 Gy fractions for e.g. breast, head, and neck cancers. According to the present invention the composition for use in treating or preventing a skin condition associated with cancer radiation therapy, the radiation treatment dose may be at least 50 Gy, preferably in the range of between 10 Gy-50 Gy, or higher.

Following the chemotherapy treatments, the inventor underwent breast mastectomy followed radiation therapy (RT) treatments. The inventor received radiation treatments for 4 weeks with a total of >50 Gy fractions. Since to date, an effective preventative measure or treatment for radiation dermatitis, or acute radiation dermatitis, is lacking yet numerous patients desperately need a solution, and for example acute radiation dermatitis may be placed in a scoring system as presented in table 1, which have been presented by the Radiation Therapy Oncology Group/European Organization for Research and Treatment of Cancer (RTOG/EORTC) toxicity criteria and National Cancer Institute Common Toxicity Criteria for Adverse Events (NCI CTCAE) systems, which is one of the most commonly used systems, showing the severity of the disease on a scale 0-5.

TABLE 1
Acute radiation dermatitis scoring

| 0 | 1 | 2 | 3 | 4 | 5 |
|---|---|---|---|---|---|
| No | Faint | Moderate | Moist | Life-threatening | Death |
| change | erythema, dry | erythema or | desquamation | consequences, | |
| | desquamation | edema, patchy | in areas other | full thickness | |
| | | moist | than skin folds, | skin necrosis/ | |
| | | desquamation, | bleeding | ulceration, | |
| | | confined to | induced by | spontaneous | |
| | | skin folds and | minor trauma | bleeding skin | |
| | | crease | | graft indicated | |

Thus, the inventor developed the composition for topical use according to the present inventive concept to fulfill this unmet need.

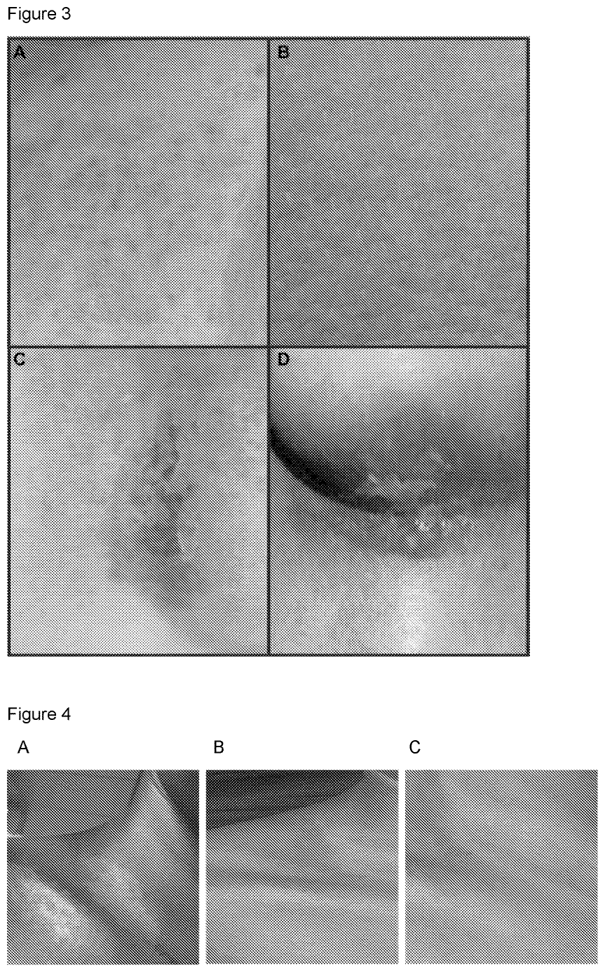

One week prior to the radiotherapy treatments, the inventor started using the topical composition according to the present invention, i.e. an example Cure-Oat or Cure-BG cream (both contain the same ingredients) 1-5 wt %., comprising colloidal oatmeal (Lantmännen) mixed with Aloe vera whole leaf extract gel, Butyrospermum Parkii (Shea) Oil in a liquid form, 4-t-butylcyclohexanol 0.5-2.0 wt %, Vitamin E, Jojoba oil, Piroctone Olamine, and white petrolatum. Then, beginning on day 1 of radiation therapy, the inventor applied the topical composition, i.e. the example Cure-Oat or Cure-BG cream three times daily, scheduled such that no cream was applied within the four hours of the scheduled radiotherapy session. After approximately 2 weeks of using the Cure-Oat or Cure-BG cream, the inventor added 1-2% of an additional beta-glucan to the cream. During the course of her radiotherapy treatment, the inventor developed mild radiation dermatitis symptoms which included redness, dryness, peeling of the skin, itching, stinging, burning, erythema, and dry moist desquamation, as shown in FIG. 3, the inventor received >50 Gy total for 4 weeks. These symptoms occur in up to 95% of patients receiving radiation therapy. However, the inventor's symptoms were mild RD compared to the patients who normally receive >50 Gy of RT fraction or higher than >50 Gy. Table 2 shows common clinical symptoms of acute radiation dermatitis which have been treated for a certain time (onset) with a certain radiation dose (Dose threshold in Gy). Thus, it was shown that the composition of the present invention is effective in alleviating symptoms of acute radiation dermatitis in association radiation therapy.

TABLE 2
Common clinical symptoms of acute radiation dermatitis

| Skin reaction | Onset | | Dose threshold (Gy) |
|---|---|---|---|
| Erythema | 7-10 | days | 6-10 |
| Dry desquamation | 3-4 | weeks | 20-25 |
| Moist desquamation | 4+ | weeks | 30-40 |
| Ulceration | 5+ | weeks | >40 |

The inventor treated herself continuously with the composition according to the invention, i.e. the example Cure-Oat or Cure-BG cream, until the skin was back to normal and there is no itching, stinging, burning, erythema, pruritus, and/or edema dry moist desquamation as shown on FIG. 4 and continued using the Cure-Oat or Cure-BG cream for 3 months. The skin in the area treated by radiotherapy stays sensitive for many years after radiotherapy treatment. The inventor uses the composition of the present invention, i.e. Cure-Oat or Cure-BG Cream, to take extra care to protect it from the sun, especially during the summertime.

### Components of the Cream

### Colloidal Oatmeal

Colloidal oatmeal is a natural product that is derived from the whole dehulled grain. It has excellent safety record and is approved by FDA (US Food and Drug Administration) as an over the counter (OTC) skin protectant. Oatmeal in colloidal form is a centuries-old topical treatment for a variety of skin conditions, including skin rashes, erythema, burns, itch, and eczema; however, few studies have investigated the exact mechanism of action for the anti-inflammatory activity of colloidal oatmeal. Additionally, oatmeal possesses potent antioxidant and anti-inflammatory properties that have shown clinical benefits in the treatment of a variety of inflammatory dermatologic disorders such as atopic dermatitis, psoriasis, and dry skin have been shown in many studies.

The active ingredients in oatmeal include polysaccharides, proteins, lipids, saponins, enzymes, flavonoids, vitamins, β-glucans, and avenanthramides. Avenanthramides are phenolic compounds present in oats that mediate its anti-inflammatory activity. Decrease activation of nuclear factor kappa B (NF-kappa B) pathway in keratinocytes and diminish the secretion of the pro-inflammatory cytokines and histamine which are well-known key mechanisms in the pathophysiology of inflammatory dermatoses, are mechanisms that could contribute to the anti-inflammatory activity of colloidal oats on inflamed, dry, and itchy skin dermatoses. Phenols are also contained in colloidal oatmeal, which possess antioxidant and anti-inflammatory activities, as well as UV-absorbing activity. Colloidal oatmeal is a FDA-approved skin protectant that temporarily protects and helps relieve minor skin irritation and itching due to rashes, eczema, poison ivy, oak, sumac, or insect bites.

Colloidal oatmeal is the finely ground whole oat kernel or groat, and is an active natural ingredient covered by the FDA OTC Skin Protectant monograph in the US. The oat grain is ground and processed until no more than 3% of the total particles exceed 150 μm and no more than 20% exceeds 75 μm. The composition of colloidal oatmeal largely consists of starch (65-85%), protein (15-20%), lipids (3-11%), fiber (5%) and β-glucans (5%).

The composition according to an embodiment of the invention includes colloidal oatmeal in a range of 1-10 wt %. Preferably the colloidal oatmeal may be in a range of 1-5 wt %.

### Avenanthramides

Avenanthramides (anthranilic acid amides) are a group of phenolic alkaloids found mainly in oats (Avena sativa), but also some other plants. Number of studies demonstrate that these natural products have antioxidant, anti-inflammatory, and anti-atherosclerotic properties, and may be used as a treatment for people with inflammatory, allergy, or cardiovascular diseases. These findings suggest that oat avenanthramides and synthetic analogs provide a broad range of health benefits that complement the already known health benefits derived from oats.

The composition according to an embodiment of the invention includes avenanthramides in a range of 0.5-5 wt %.

### Beta-Glucans (β-Glucans)

Historically, investigations into the phytochemical constituents of oat have focused primarily on their value as a food. For example, β-glucan is the “soluble fiber” that makes oats a heart-healthy food. β-glucans have also been used as scaffolds for the growth of bioartificial skin, and are known to assist in wound healing, response to injury and infection, and have a great water retention capacity.

β-Glucans, which are glucose polymers, may be from a variety of sources including yeast, grain, and fungus, belong to the class of drugs known as biological response modifiers. Accordingly, β-glucans form a natural component of the cell walls of bacteria, fungi, yeast, and cereals such as oat and barley. Each type of beta-glucan comprises a different molecular backbone, level of branching, and molecular weight which affects its solubility and physiological impact. One of the most common sources of β(1,3)D-glucan for supplement use is derived from the cell wall of baker's yeast (Saccharomyces cerevisiae). β-glucans found in the cell walls of yeast contain a 1,3-carbon backbone with elongated 1,6-carbon branches. Other sources include seaweed, and various mushrooms, such as lingzhi, shiitake, chaga, and maitake, which are under preliminary research for their potential immune effects.

β-Glucans comprise a group of β-D-glucose polysaccharides naturally occurring in the cell walls of cereals, bacteria, protozoa, and fungi, with significantly differing physicochemical properties dependent on source. At dietary intake levels of at least 3 g per day, oat fiber β-glucan decreases blood levels of LDL cholesterol and so may reduce the risk of cardiovascular diseases. Depending upon the source and method of isolation, beta-glucans have various degrees of branching and type of linkage in the backbone and side chains.

Numerous studies have shown that β-D-glucans, either particulate or soluble, enhance immune functions with anti-infective, antitumor and immunomodulatory activity. All β-glucans are glucose polymers linked by 1,3-; 1,4- or 1,6-β-glycosidic bonds and differ from each other by their length and branching structure. The biological actions of the different β-glucans vary according to their molecular structure, solubility and the conformation of each polymer.

One promising area of β-glucan application is dermatology, including wound care. Topical application of β-glucans is increasing, since their pluripotent activity antioxidant, anti-inflammatory and regenerative effects, immunomodulation, radioprotection, moisturization and rejuvenation—might help as a complementary therapy in managing various skin diseases and conditions. In addition, some β-glucans also possess anti-infective properties and exhibit potential antibacterial activity against a broad spectrum of Gram-positive and Gram-negative bacteria. β-Glucans may therefore represent a suitable wound healing-agent with great stability and a broad range of biological activities. Wound healing is a complex process involving various cellular and extracellular matrix components and cells (keratinocytes, fibroblasts, endothelial cells, mast cells, nerve cells and leucocyte subtypes) that participate differently in the three overlapping phases (inflammation, cell proliferation and tissue remodeling). As mentioned above, the recognition of and response to β-glucans are mediated primarily by cell surface receptors, including those from immunocytes and cutaneous cells. In terms of β-glucan immunostimulatory activity regarding wound healing, two modes of action are possible. One manifests by indirect activation through various cytokines of macrophages and the other by direct influence on keratinocytes and fibroblasts.

The composition according to an embodiment of the invention comprises β-glucans 1-10% by wt, in powder form. A preferred composition comprises colloidal oatmeal and β-glucan 1-5% by wt in 50 ml of cream.

### Avena Sativa (Oat) Kernel Oil

Avena Sativa (oat) kernel oil or oat kernel oil is a lipid extracted from oats with considerable moisturizing and healing properties and a non-greasy finish. A fast-absorbing oil packed with linoleic fatty acid, sterols, ceramides and sphingolipids, antioxidants and anti-inflammatory agents, oat kernel oil is non-comedogenic and can even reduce the appearance of papules, blackheads, and lesions, making it a great choice for acne prone skin types. Oat kernel oil has high polar lipid content including ceramides and acts as an occlusive and a humectant, absorbing and retaining moisture in the skin. Topical oat oil application has been shown to increase ceramide content in skin by up to 70%. Oat kernel oil has also shown effectiveness in treating a variety of skin conditions, including skin rashes, erythema, burns, itch, and eczema thanks to its anti-inflammatory abilities.

Oat kernel oil is rich in fatty acids similar to those of the skin, including phospholipids and ceramides. It has a high content of linoleic acid, which improves the protective barrier function of the skin, and antioxidants, such as tocopherols and tocotrienols, which protect the skin's lipids from damage caused by UV rays. It has excellent absorption properties, strengthens the skin feel, moisturizes and soothes the skin.

Oat kernel oil is a separate component from avenanthramides and colloidal oats. Oats are generally regarded as having the highest lipid content of any cereal grain (^{~}7% to 10%) and as an excellent source of unsaturated fatty acids. Oat oil is a mixture of lipid components, each with its own biological properties. When fractionated, oat oil is found to be composed of around 50% triglycerides, 14% mono- and diglycerides, and 10% free fatty acids, with smaller amounts of sterols, phosphatidyl choline, phosphatidyl ethanolamine, and other compounds, all of which are affected to some extent by cultivation conditions, plant genetics, and the method of lipid extraction. Oat lipids contain about 80% unsaturated fatty acids, which in turn are about 42% to 52% linoleic acid. Linoleic acid has been shown effective in reducing trans epidermal water loss and restoring the skin permeability barrier.

4-t-Butylcyclohexanol

4-tert-butylcyclohexanol, or 4-t-butylcyclohexanol, is a synthetic fatty alcohol (the non-irritating kind) that functions as an emollient and soothing agent. It can reduce skin surface stinging on contact and is also capable of visibly reducing redness on skin. In addition to its general skin-calming properties, 4-t-butylcyclohexanol has specifically been studied for its ability to effectively reduce shaving-induced skin irritation. Dedicated research has also been conducted to demonstrate its efficacy for improving other aggravated skin conditions around the eyes and mouth. Essentially, it works by intercepting the irritation signals in skin. 4-t-butylcyclohexanol is an effective active ingredient concept for the threatening with of sensitive skin, as the topical application resulted in an immediate relief from symptoms such as erythema and stinging. A soothing molecule whose special feature is to instantly decrease stinging and burning sensations, it can reduce stinging and burning sensation quickly after application. It is so effective because it works at a cellular level via intercepting the signals caused by the irritation before they can reach the neuro-receptor. This means that the stinging and burning sensations are less pronounced and the skin's tolerance threshold is increased.

Cosmetic compositions for reducing human skin irritation, preferably suitable for topical application on human skin, comprise 4-t-butylcyclohexanol 0.1-4.5 wt %, preferably 0.25-4.0 wt %, more preferably 0.375-3.0 wt %, even more preferably 0.5-2.5 wt %, most preferably 0.6-2 wt %, of trans-4-t-butylcyclohexanol or a cosmetically or pharmaceutically acceptable salt thereof, in particular the Na+, K+, NH 4+, Mg 2+ or Ca 2+ salt, based on the total weight of the composition.

Composition according to an embodiment of the invention comprises wherein the weight ratio of trans-4-tert-butyl cyclohexanol to cis-4-tert-butyl cyclohexanol, if present, is 70:30 or greater, preferably greater than 85:15, more preferably greater than 90:10, most preferably greater than 95:5.

The composition according to an embodiment of the invention comprises 4-t-butylcyclohexanol 0.1-5 wt % in solid form mixed in 50 ml of the composition.

### Piroctone Olamine

Piroctone olamine (1-hydroxy-4-methyl-6-(2,4,4-trimethyl)-2-(1H)pyridinone,2-aminoethanol salt) is an ethanolamine salt of the hydroxamic acid derivative piroctone. First synthesized in 1979 by Schwarzkopf-Henkel (Germany), this antifungal compound is a component of many cosmetic products such as anti-dandruff shampoo and hair rinses for scaly and irritated skin due to the colonization with Malassezia spp, and other yeasts. These microorganisms split parts of the sebum of the capillitium lipolytically into free fatty acids. These free fatty acids and microbial peroxidases lead to irritation of the skin and via an increase of mitosis to scaling. The piroctone olamine (PO) present in the shampoo and hair rinses have the ability to reduce microbial colonization. Its mechanism of action is complex and not completely understood. It is known that this agent has the ability to penetrate the cell membrane and form complexes with irons (Fe2+e Fe3+), inhibiting energy metabolism in mitochondria of pathogenic fungi.

Piroctone olamine is a hydroxamic acid that inhibits ergosterol synthesis, the main component of the cell wall of most fungi. It is used as a cream or shampoo at a 0.5-1% concentration. Piroctone olamine alone or in combination with other agents has been demonstrated to reduce erythema, scaling, burning/stinging sensation, and pruritus in fungal disease, with excellent antioxidant technologies.

Piroctone olamine has been approved for use in cosmetic products at a maximal concentration of 1% (rinse-off products) or 0.5% in other products according to “Cosmetic products marketed in the EU may only contain those preservatives which are listed in Annex VI of the Cosmetics Directive 76/768/EEC, “List of preservatives which cosmetic products may contain”.

The composition according to an embodiment of the invention comprises piroctone olamine 0.1-5 wt %, in white powder form.

### White Petrolatum

White Petrolatum is used as an ointment base, topical skin protectant, and lubricant. It temporarily relieves minor, cuts, scrapes, burns, chapped or cracked skin and lips. Additionally, it helps protect from the effects of wind and cold weather.

In June of 2003 the FDA published the “Final Rule” in the Federal Register 7 that listed petrolatum as a skin protectant as long as the concentration was in the range of 30% to 100%. Petrolatum is often not fully refined in the US, which means it can be contaminated with toxic chemicals called polycyclic aromatic hydrocarbons (PAHs).

In the present compounds, white petrolatum is used 0.1-20 wt %, in the form of a white gel. A preferred embodiment comprises 10% in 50 ml in white petrolatum/paraffin in the form of a white gel.

### Aloe Vera Gel

The Aloe vera plant produces two substances used in health care products-clear gel and yellow latex. It is primarily used aloe vera clear gel topically in creams and ointments to treat burns, the skin condition psoriasis and even acne or other skin conditions.

Research on the use of aloe vera for specific conditions shows that the application of aloe gel appears to shorten the duration of wound healing for first- and second-degree burns and might also promote wound healing. For acne, aloe gel, applied in the morning and evening in addition to the use of the topical prescription acne medicine tretinoin (Retin-A, Atralin, others), might be more effective in reducing acne than using a topical prescription alone.

For psoriasis, aloe extract cream might reduce redness, scaling, itching and inflammation caused by mild to moderate psoriasis. For the Herpes simplex virus, applying a cream containing aloe extract might help lesions heal sooner. The composition according to an embodiment of the invention comprises aloe vera gel 0.1-5 wt %, preferably 2-5 wt % as a yellow fade to white gel.

Butyrospermum parkii

Butyrospermum parkii (shea) use has increased from 2011 to 2018, taking the first place as the most used botanical. Shea is mainly used for its butter, which consists of a solid fat extracted from mature shea fruit. It contains 90% triglycerides (saponifiable fraction) and 10% non-triglycerides (unsaponifiable fraction). The main fatty acids found in shea are stearic, oleic, palmitic, linoleic, and arachidic acid, which provide moisturizing and barrier protective actions. The unsaponifiable include antioxidants (oil soluble tocopherols), triterpenes (e.g., butyrospermol), phenols, sterols, karitene, allantoin, and polyphenols (mainly the catechin), which together have been shown to provide UV-B absorbing properties. Shea butter has shown to boost collagen production while inactivating proteases such as metalloprotease (e.g., collagenase) as well as serine protease (e.g., elastase). Two clinical studies showed that shea butter is able to reduce multiple signs of aging and prevent photo-aging. Aside from “Butyrospermum parkii (shea) butter”, whose use almost tripled in 2018, one product containing “Butyrospermum parkii (shea) Butter Extract” was also found in 2018, which contains a higher bioactive fraction of shea butter triterpene esters. The evidence regarding shea butter advantages for skin aging may justify the increased use in anti-aging products from 2011 to 2018 as well as the development of differentiated preparations.

The composition according to an embodiment of the invention comprises Butyrospermum parkii in the range of 0.1-5 wt %, preferably 2-5 wt % in the form of a colorless oil.

### Vitamin E

Vitamin E is a lipophilic antioxidant known for its beneficial effects on the skin, including photoprotection, hydration, and accelerated epithelialization, among others.

Vitamin E is a family of lipid-soluble compounds consisting of two major groups, namely, tocopherols and tocotrienols. Each group can be further divided into four different isomers, that is, alpha, beta, gamma, and delta, depending on the presence and position of the methyl group(s) as the side chain. Tocopherols have a long and saturated side chain, while tocotrienols differ from tocopherols by the presence of unsaturated double bonds on the side chain. This also explains the higher affinity of tocotrienols to the lipid membrane compared to tocopherols.

Vitamin E has gained researchers' attention as a potential adjuvant therapy for various disorders due to its excellent antioxidant and anti-inflammatory properties. Inflammation is a result of the enhanced body immune response and is characterized by the increased production of free radicals and pro-inflammatory mediators, which may lead to excessive damage to host tissues. With regard, to the anti-inflammatory properties of vitamin E, several studies have demonstrated that vitamin E could inhibit the secretion of inflammation-mediating molecules such as eicosanoids and cyclooxygenase-2 enzyme (COX-2). Vitamin E also suppresses pro-inflammatory signaling pathways, such as nuclear factor kappa beta (NF-kβ) and signal transducer and activator of transcription 3 (STAT-3)-mediated pathways.

Unfortunately, the bioavailability of vitamin E is poor. Current research aims to make vitamin E available in nano-formulations, which would increase its absorption, cellular uptake, and stability.

The composition according to an embodiment of the invention comprises natural vitamin E oil (1000 IU) 0.1-2 wt %, USP 10 ppm maximum. In a preferred embodiment, 1-3 drops vitamin E oil in the form of a yellow to yellowish brown, clear and viscous liquid, were mixed in 50 ml of the composition.

### Jojoba Oil

Jojoba is a widely used medicinal plant that is cultivated worldwide. Its seeds and oil have a long history of use in folklore to treat various ailments, such as skin and scalp disorders, superficial wounds, sore throat, enhancement of immunity, and promotion of hair growth. Extensive studies on Jojoba oil showed a wide range of pharmacological applications, including antioxidant, anti-acne and anti-psoriasis, anti-inflammatory, antifungal, antipyretic, analgesic, antimicrobial, and anti-hyperglycemia activities. In addition, Jojoba oil is widely used in the pharmaceutical industry, especially in cosmetics for topical, transdermal, and parenteral preparations. Jojoba oil also holds value in the industry as an anti-rodent, insecticides, lubricant, surfactant, and a source for the production of bioenergy. Jojoba oil is considered among the top-ranked oils due to its wax, which constitutes about 98% (mainly wax esters, few free fatty acids, alcohols, and hydrocarbons). In addition, sterols and vitamins with few triglyceride esters, flavonoids, phenolic and cyanogenic compounds are also present.

Jojoba oil in single-phase and emulsion systems shows an excellent lubricity without the oily, greasy feel of other lipids, especially lanolin and petrolatum. It can also contribute to superior transpirational water control in the skin, thus reducing evaporation without blocking the passage of gases and water vapor. This character is due to its high molecular weight and low viscosity, and structural similarity with skin sebum, leading to a smoothing effect on dry skins and the inhibition of excess flaking of epidermal cells [5,33]. Skin indentation tests showed that the oil enhanced skin elasticity, similar to the effect of lanolin. Jojoba oil also showed a keratoplastic effect and seemed to restore the skin's natural shine. Jojoba oil contains antioxidants, such as vitamins A and E, and omega-6 fatty acids, which are essential components of the skin's barrier. It also acts as a humectant, meaning it works to retain moisture in the skin. This protective layer, combined with the anti-inflammatory effects of the vitamin E, gives skin the breather necessary to focus on the healing process.

The composition according to an embodiment of the invention comprises Jojoba oil 0.1-2 wt %, preferably approximately 1 wt % in the form of a clear golden liquid.

### Preservatives and Other Components

The compositions may comprise various components known to increase the shelf-life of the components. For example, ascorbic acid may be used in 0.1 wt %. to prevent immediate oxidation when filling bottles. Alternatively, or additionally, the compositions may contain Cosphaderm LA-T (Levulinic Acid, Glycerin, Sodium Levulinate, Aqua) 0.5-1 wt %, and/or natural preservative E-202 (potassium sorbate) in approximately 0.1 wt %.

Ethylhexylglycerin, approximately 0.2 wt % may be used as a preservative, surfactant, deodorizer, and a skin-conditioning agent.

### Emollients

The compositions herein described may further comprise emollients such as Caprylic/Capric triglyceride and/or evening primrose in pharmaceutically acceptable dosages.

Caprylic/Capric triglyceride is an effective emollient and skin conditioner that leaves the skin smooth and supple.

Evening primrose (Oenothera biennis) is a wild plant of medicinal importance; its seed oil is traditionally used for treatment of eczema, asthma, rheumatoid arthritis, premenstrual and menopausal syndrome, and other inflammation-related disorders. Evening primrose oil is a non-fragrant plant oil whose fatty acids can have significant skin-soothing and emollient benefits for the skin and may also aid in the absorption of other ingredients into the skin.

Hydrogenated vegetable oil is used an emollient in cosmetic formulations to soften skin/hair. It also helps enhance hydration by forming a barrier on skin's surface that reduces moisture loss. This ingredient is created by adding hydrogen atoms to vegetable oils through a process known as hydrogenation.

### pH Adjusters

The compositions may comprise one of more of the following compounds for adjusting the pH: citric acid monohydrate (pH adjuster), sodium citrate (pH adjuster), and/or sodium hydroxide (pH adjuster).

### Natural Thickeners

The compositions may comprise xanthan gum, sodium phytate, and or sclerotium gum for thickening and/or improving the texture of the compositions. Xanthan gum, a natural thickener extracted from corn, may be used in approximately 0.1 wt %.

Sodium phytate is the synthetic salt form of the antioxidant phytic acid. In this form, it works as a chelating agent, keeping metals from binding to water in cosmetics; it may be used in approximately 0.01 wt %.

0.3% Sclerotium gum is used in cosmetics to thicken and stabilize formulas. It also functions as a skin conditioning agent. Sclerotium gum is also used as a natural alternative to synthetic gelling agents such as carbomer. The Cosmetics Ingredient Review board has deemed multiple times that sclerotium gum is considered safe as used in cosmetics, where it's typical concentration ranges from 0.25-2%, depending on the desired product aesthetics and needs of ingredients it's blended with. Preferably, the compositions may comprise sclerotium gum in 0.3 wt %.

### Experimental Data & Further Investigations

### Radiation Dermatitis and Hand-Foot-Syndrome—In Vitro 3D Cell Tests

Skin is the first barrier against external aggressions and is therefore the first target of many environmental stressors, such as radiation therapy. Those deleterious factors are correlated with skin ageing, inflammation and allergic reactions.

In this study, we will investigate a wide panel of functional and transcriptomic assays evaluating the protective and repairing effect of ingredients against radiation therapy and chemotherapy damages. We will use UV induction as a single dose 3D keratinocyte or chemically induced the cells. The composition for topical use according to the present inventive concept will be applied before or after these doses.

Expected Results: We will use TaqMan Low Density RT-qPCR 96 genes Array to study the expression of key genes involved in cell response to radiation damage. It is expected that the treated cells with the composition for topical use of Cure-Oat or Cure-BG cream according to the present inventive concept will show effective properties to repair or prevent from adverse events of radiation dermatitis, HFS, reduce inflammatory and oxidative response, and to detoxify, and repair the dermal extracellular matrix.

### Clinical Study: A Study of Cure-Oat or Cure-BG in the Prevention of Radiation Dermatitis in Breast Cancer Patients

Inclusion Criteria: 1. Female patients 2. Invasive breast cancer confirmed by pathology 3. Underwent mastectomy and/or breast reconstruction 4. Radiotherapy after breast reconstruction. Patients who might develop RD grade ⅔ between and receiving total fractions of 10-50 of radiation therapy with all locations and >50 fractions. ARM I Group: Patients apply Cure-Oat or Cure-BG topically to the affected area tree times daily starting at least one week prior the first dose of radiation therapy until dermatitis has returned to normal. ARM II Group: Patients will receive placebo.

Expected Results: Colloidal oatmeal, a natural product with proven barrier protection, moisturization, anti-inflammatory, and soothing properties. We will compare patients who will be using Cure-Oat or Cure—BG cream to the placebo group and skin toxicities between groups. Cure-Oat or Cure-BG cream will significantly reduce skin toxicity from RT, while the placebo group will show moderate/severe symptoms of RD. Breast cancer patients treated with the placebo will developed significantly higher rate of ≥grade 2 dermatitis than did skin treated with Cure-Oat or Cure-BG cream as indicated on FIG. 4.

Clinical Study of Cure-Oat or Cure-BG in the Prevention of Hand-Foot-Syndrome in Patients with Metastatic Breast Cancer

Inclusion Criteria: 1. Patients with metastatic breast cancer who are scheduled to receive chemotherapies treatments. Patients will apply Cure-Oat or Cure-BG topically to the affected area tree times daily starting at least one week prior the first dose of chemotherapies treatments and continue until HFS has returned to normal.

Expected Results: Colloidal oatmeal possesses potent antioxidant and anti-inflammatory properties that its clinical benefits in the treatment of a variety of inflammatory dermatologic disorders such as atopic dermatitis, psoriasis, and dry skin have been shown in the previous clinical studies. We will compare patients who will be using Cure-Oat or Cure-BG to the placebo group and skin toxicities between the groups. It is expected that Cure-Oat or Cure-BG t will significantly reduce skin toxicity from HFS, while the placebo group will show moderate/severe symptoms of HFS. Breast cancer patients treated with the placebo will likely develop significant blisters, cracks and sores on their hands and feet compared to patients treated with Cure-Oat or Cure-BG cream.

### The Effect of Radiation Dermatitis and Hand-Foot-Syndrome on Skin Microbiome

Our skin is home to millions of bacteria, fungi and viruses which compose the skin microbiota. Skin microbiota interacts with skin and affects physiology and immunity of the skin. Previous studies have reported the effects of radiotherapy or chemotherapy on skin and immune system. However, so far, no studies have analyzed the effects of radiotherapy or chemotherapy on skin microbiome and how skin microbiome affects the skin immune responses after chemotherapy or radiotherapy treatments. In these claims, the breast cancer patients who is receiving chemotherapy or radiotherapy treatments will be enrolled.

Criteria: The breast cancer patients who will receive chemotherapy and radiotherapy will be enrolled. The skin microbiome before chemotherapy treatments and radiotherapy and its changes after chemotherapy/radiotherapy will be analyzed systematically to find out whether the skin microbiome is associated with the severity of radiation dermatitis and HFS. Patients will apply Cure-Oat or Cure-BG topically to the affected area three times daily starting at least one week prior the first dose of chemotherapies/RT treatments and continue until the skin has returned to normal. The reason why it is necessary to start one week prior the first dose of chemotherapy and radiotherapy is that β-glucans possess anti-infective properties and exhibit potential antibacterial activity against a broad spectrum of Gram-positive and Gram-negative bacteria and possibly to balance skin microbiome. The long-term goal of this claim is to deepen the understanding of the role of skin microbiome in the occurrence and development of radiation dermatitis/HFS and provide a basis for subsequent exploration to reduce radiation dermatitis and HFS by regulating skin microecology. It will be used to analyze these samples Next-Generation sequencing.

Expected Results: We hypothesize that RT and chemotherapy treatments decrease diversity of the skin microbiome and an increasing pathogenic skin microbes (e.g., S. aureus) with the emergence of skin toxicities. Additionally, we hypothesize that chronic skin inflammation of RD and HFS play mechanistic role in the skin microbiome-related treatment toxicities.

Therefore, the treatment of Cure-Oat or Cure-BG will minimize skin toxicity and keep skin microbiome in balance which will be potential interventions for preventing and treating skin toxicities among patients with cancer.

### ITEMIZED LIST OF EMBODIMENTS

Item 1. A composition for topical administration comprising:
a colloidal oatmeal and a 4-t-butylcyclohexanol, wherein the colloidal oatmeal comprises at least one of β-glucan and avenanthramide.

Item 2. The composition for topical administration according to item 1, wherein the colloidal oatmeal is in an amount in the range of 1-10 wt % of the total amount of the composition, preferably in the range of 1-5 wt %.

Item 3. The composition for topical administration according to item 1 or 2, wherein the 4-t-butylcyclohexanol is in an amount in the range of 0.1-5 wt % of the total amount of the composition, preferably in the range of 0.5-2.0 wt %.

Item 4. The composition for topical administration according to anyone of items 1-3, further comprises additional β-glucan in the form of a liquid or powder, preferably in an amount in the range of 1-10 wt % of the total amount of the composition, preferably in the range of 1-5 wt %.

Item 5. The composition for topical administration according to anyone of items 1-4, further comprising at least one of Aloe vera whole leaf extract, Butyrospermum Parkii Shea oil, avenanthramides, white petrolatum, vitamin E, Jojoba oil, and piroctone olamine.

Item 6. The composition for topical administration according to anyone of items 1-5, further comprising one or more additional components selected from dermatologically acceptable carriers, preservatives, surfactants, humectants, emulsifiers, thickening agents, perfumes, preservatives, vegetable or mineral oils, antiseptic agents, acidifying or alkalinizing agents, vitamins, antioxidants, anti-UV agents, solvents, pH-stabilizing agents, silicones and combinations thereof.

Item 7. The composition for topical administration according to any one of items 1-6, comprising:
1-10 wt % colloidal oatmeal;
0.1-5 wt % 4-t-butylcyclohexanol;
optionally 1-10 wt % β-glucan;
0.1-5 wt % piroctone olamine;
0.1-5 wt % Aloe vera whole leaf extract;
0.1-2 wt % Butyrospermum Parkii Shea oil;
optionally 0.5-5 wt % avenanthramides;
0.1-20 wt % white petrolatum; and
20-60 wt % water.

Item 8. A composition for topical administration according to anyone of items 1-7 for use in therapy.

Item 9. A composition for topical administration according to anyone of items 1-7 for use in treating or preventing a skin condition associated with cancer radiation therapy, chemotherapy or sunburn.

Item 10. The composition for use according to item 9, wherein the skin conditions are selected from the hand-foot syndrome, blisters, dry or moist desquamation, redness, dryness, and/or peeling of the skin, itching, stinging, burning, erythema, atopic dermatitis, psoriasis, inflammation, irritation and allergy.

1. A composition for topical administration comprising:
a colloidal oatmeal and a 4-t-butylcyclohexanol, wherein the colloidal oatmeal comprises β-glucan and optionally avenanthramide, wherein the 4-t-butylcyclohexanol is in an amount in the range of 0.1-5 wt % of the total amount of the composition, and wherein the β-glucan is in an amount in the range of 1-10 wt % of the total amount of the composition.

2. The composition for topical administration according to claim 1, wherein the colloidal oatmeal is in an amount in the range of 1-10 wt % of the total amount of the composition, preferably in the range of 1-5 wt %.

3. The composition for topical administration according to claim 1, wherein the 4-t-butylcyclohexanol is in an amount in the range of 0.5-2.0 wt % of the total amount of the composition.

4. The composition for topical administration according to claim 1, comprises β-glucan in an amount in the range of 1-5 wt % of the total amount of the composition.

5. The composition for topical administration according to claim 1, further comprises β-glucan in the form of a liquid or a powder.

6. The composition for topical administration according to claim 1, wherein the composition is a form of a lotion, gel, cream, ointment, or spray.

7. The composition for topical administration according to claim 1, further comprising at least one of Aloe vera whole leaf extract, Butyrospermum Parkii Shea oil, avenanthramides, white petrolatum, vitamin E, Jojoba oil, and piroctone olamine.

8. The composition for topical administration according to claim 1, further comprising one or more additional components selected from dermatologically acceptable carriers, preservatives, surfactants, humectants, emulsifiers, thickening agents, perfumes, preservatives, vegetable or mineral oils, antiseptic agents, acidifying or alkalinizing agents, vitamins, antioxidants, anti-UV agents, solvents, pH-stabilizing agents, silicones and combinations thereof.

9. The composition for topical administration according to claim 1, comprising:
1-10 wt % colloidal oatmeal;
0.1-5 wt % 4-t-butylcyclohexanol, preferably 0.5-2.0 wt %;
1-10 wt % β-glucan, preferably 1-5 wt %;
0.1-5 wt % piroctone olamine;
0.1-5 wt % Aloe vera whole leaf extract;
0.1-2 wt % Butyrospermum Parkii Shea oil;
optionally 0.5-5 wt % avenanthramides;
0.1-20 wt % white petrolatum; and
20-60 wt % water.

10. A composition for topical administration according to claim 1 for use in therapy.

11. A composition for topical administration according to claim 1 for use in treating or preventing a skin condition associated with cancer radiation therapy or chemotherapy.

12. The composition for use according to claim 11, wherein the skin conditions are selected from hand-foot syndrome, blisters, dry or moist desquamation, redness, dryness, peeling of the skin, itching, stinging, burning, erythema, atopic dermatitis, psoriasis, inflammation, irritation and radiation dermatitis.

13. A composition for topical administration according to claim 1 for use in treating or preventing a skin condition associated with cancer radiation therapy.

14. The composition for use in treating or preventing a skin condition associated with cancer radiation therapy according to claim 13, wherein the skin condition is radiation dermatitis.

15. The composition for use in treating or preventing a skin condition associated with cancer radiation therapy according to claim 13, wherein the radiation treatment dose is at least 50 Gy, preferably the radiation treatment dose is in the range of 10 Gy-50 Gy.

16. A composition for topical administration according to claim 1 for use in treating or preventing a skin condition associated with chemotherapy.

17. The composition for use in treating or preventing a skin condition associated with chemotherapy according to claim 16, wherein the skin conditions is hand-foot syndrome.

18. Use of a composition for topical administration according to claim 1 in treating or preventing a skin condition associated with cancer radiation therapy or chemotherapy.

19. The use of a composition for topical administration according to claim 18, wherein the skin condition is selected from the hand-foot syndrome, blisters, dry or moist desquamation, redness, dryness, peeling of the skin, itching, stinging, burning, erythema, atopic dermatitis, psoriasis, inflammation, irritation and radiation dermatitis.

20. The use of a composition for topical administration according to claim 18,
wherein the treating or preventing a skin condition is associated with cancer radiation, preferably the skin condition is radiation dermatitis; or
wherein the treating or preventing a skin condition is associated with chemotherapy, preferably the skin condition is hand-foot syndrome.

21. (canceled)
